(19) United States
(12) Patent Application Publication
(10) Pub. No.: US 2021/0186733 A1
(43) Pub. Date: Jun. 24, 2021

(54) RECTAL INSERTION DEVICE

(71) Applicant: AVINITI, LLC, Los Angeles, CA (US)

(72) Inventors: May Mahboubeh MIRAIE ASHTIANI, Stuttgart (DE); Leon MARKOVIC, Stuttgart (DE)

(73) Assignee: AVINITI, LLC, Los Angeles, CA (US)

(21) Appl. No.: 17/195,707

(22) Filed: Mar. 9, 2021

Related U.S. Application Data
(63) Continuation of application No. PCT/IB2019/058738, filed on Oct. 14, 2019.
(60) Provisional application No. 62/747,409, filed on Oct. 18, 2018.

(51) Int. Cl. A61F 5/00

(52) U.S. Cl. CPC A61F 5/0093

(57) ABSTRACT

A rectal insertion device for relieving gas in a human or animal may include a body coupled to a shaft. A conduit may extend through the body and the shaft. A nose may be coupled to the shaft via one or more nose arms, and the nose may be opposingly positioned to the body. One or more shaft apertures may preferably be formed by portions of the nose arms and nose, and each shaft aperture may be in communication with the conduit. A stopper may be coupled to the shaft, and the stopper may surround a portion of the shaft. The shaft may include one or more grooves which may be depressed into the shaft, and the stopper may include one or more stopper apertures which may extend through the stopper. Preferably, each groove may be aligned with a stopper aperture.

### CROSS REFERENCE TO RELATED APPLICATIONS

This application claims priority to international patent application no. PCT/IB2019/058738 filed on Oct. 14, 2019 which claims priority to and the benefit of the filing date of U.S. Provisional Application No. 62/747,409, filed on Oct. 18, 2018, entitled “RECTAL INSERTION DEVICE”, which is hereby incorporated by reference in its entirety.

### FIELD OF THE INVENTION

This patent specification relates to the field of devices for relieving human or animal gas. More specifically, this patent specification relates to a device for suitable for rectal insertion to provide gas relief.

### BACKGROUND

Gas is a natural byproduct of digestion and is normal for humans and animals to pass gas (flatus) through the rectum, although the volume and frequency may vary greatly between individuals. Gas is brought to the rectum via contractions of the muscles in the intestines and colon. Unfortunately, some individuals have trouble releasing gas from the rectum. This can lead to unpleasant symptoms such as pain, bloating and abdominal distension. This can be especially troublesome for infants and young children as they lack understanding and communication skills. While some devices exist for providing gas relief via rectal insertion, they are not only difficult and expensive to produce, but they can easily damage the delicate tissue of the anal canal resulting in severe injury.

Therefore, a need exists for novel devices for relieving gas in a human or animal. There is also a need for novel devices for suitable for rectal insertion to provide gas relief. A further need exists for novel devices for relieving gas in a human or animal that are not difficult and expensive to produce. Finally, a need exists for novel devices for relieving gas in a human or animal that are safe and able to prevent damage to the anal canal.

### BRIEF SUMMARY OF THE INVENTION

A rectal insertion gas relief device is provided. In some embodiments, the device may include a body that may be coupled to a shaft. The body may have a distal end and the shaft may have a proximal end. A conduit may extend between the distal end of the body and the proximal end of the shaft. A nose may be coupled to the proximal end of the shaft via one or more nose arms. One or more shaft apertures may be in communication with the conduit, and portions of one or more of the shaft apertures may be formed by the nose arms, nose, and/or proximal end of the shaft. A stopper may be coupled to the shaft, and the stopper may extend above the shaft. The device may be used by inserting the shaft into the anus of an individual with the insertion limited by the stopper. Gas may exit the individual by optionally passing through the conduit.

In further embodiments, the shaft may include one or more grooves which may be depressed into the shaft, and the stopper may include one or more stopper apertures which may extend through the stopper. Preferably, each groove may be aligned with a stopper aperture. Gas may exit the individual by optionally passing in or through a groove and out through a stopper aperture.

### BRIEF DESCRIPTION OF THE DRAWINGS

Some embodiments of the present invention are illustrated as an example and are not limited by the figures of the accompanying drawings, in which like references may indicate similar elements and in which:

FIG. 1 depicts a front perspective view of an example of a rectal insertion device according to various embodiments described herein.

FIG. 2 illustrates a side perspective view of an example of a rectal insertion device according to various embodiments described herein.

FIG. 3 shows a front elevation view of an example of a rectal insertion device according to various embodiments described herein.

FIG. 4 depicts a rear elevation view of an example of a rectal insertion device according to various embodiments described herein.

FIG. 5 illustrates a sectional elevation view of an example of a rectal insertion device according to various embodiments described herein.

FIG. 6 shows a side elevation view of an example of a rectal insertion device according to various embodiments described herein.

### DETAILED DESCRIPTION OF THE INVENTION

The terminology used herein is for the purpose of describing particular embodiments only and is not intended to be limiting of the invention. As used herein, the term “and/or” includes any and all combinations of one or more of the associated listed items. As used herein, the singular forms “a,” “an,” and “the” are intended to include the plural forms as well as the singular forms, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof.

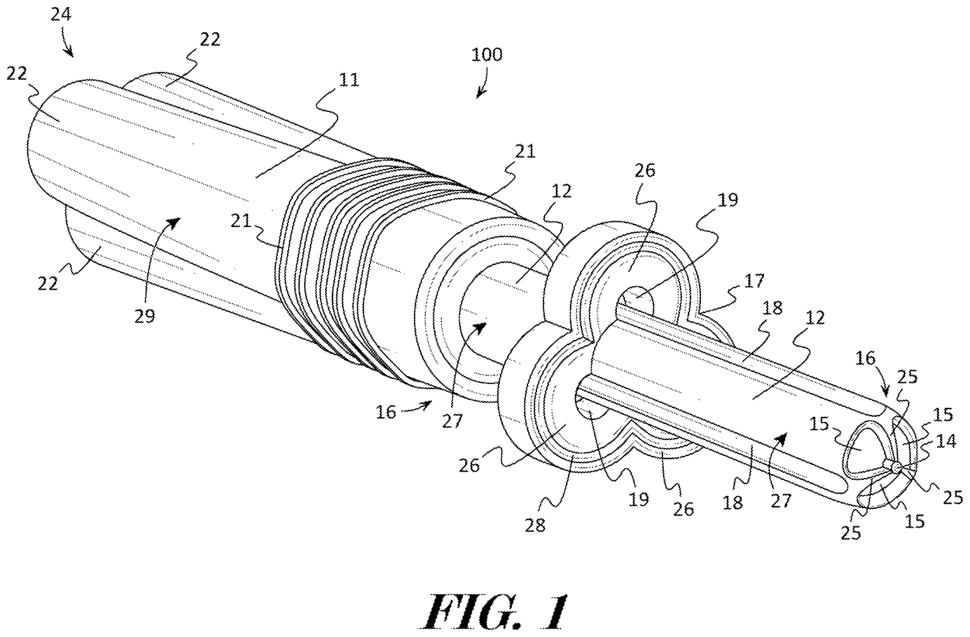

For purposes of description herein, the terms “upper”, “lower”, “left”, “right”, “rear”, “front”, “side”, “vertical”, “horizontal”, and derivatives thereof shall relate to the invention as oriented in FIG. 1. However, one will understand that the invention may assume various alternative orientations and step sequences, except where expressly specified to the contrary. Therefore, the specific devices and processes illustrated in the attached drawings, and described in the following specification, are simply exemplary embodiments of the inventive concepts of the invention. Hence, specific dimensions and other physical characteristics relating to the embodiments disclosed herein are not to be considered as limiting, unless expressly stated otherwise.

Although the terms “first”, “second”, etc. are used herein to describe various elements, these elements should not be limited by these terms. These terms are only used to distinguish one element from another element. For example, the first element may be designated as the second element, and the second element may be likewise designated as the first element without departing from the scope of the invention.

As used in this application, the term “about” or “approximately” refers to a range of values within plus or minus 10% of the specified number. Additionally, as used in this application, the term “substantially” means that the actual value is within about 10% of the actual desired value, particularly within about 5% of the actual desired value and especially within about 1% of the actual desired value of any variable, element or limit set forth herein.

Unless otherwise defined, all terms (including technical and scientific terms) used herein have the same meaning as commonly understood by one having ordinary skill in the art to which this invention belongs. It will be further understood that terms, such as those defined in commonly used dictionaries, should be interpreted as having a meaning that is consistent with their meaning in the context of the relevant art and the present disclosure and will not be interpreted in an idealized or overly formal sense unless expressly so defined herein.

In describing the invention, it will be understood that a number of techniques and steps are disclosed. Each of these has individual benefit and each can also be used in conjunction with one or more, or in some cases all, of the other disclosed techniques. Accordingly, for the sake of clarity, this description will refrain from repeating every possible combination of the individual steps in an unnecessary fashion. Nevertheless, the specification and claims should be read with the understanding that such combinations are entirely within the scope of the invention and the claims.

A new device for relieving gas in a human or animal is discussed herein. In the following description, for purposes of explanation, numerous specific details are set forth in order to provide a thorough understanding of the present invention. It will be evident, however, to one skilled in the art that the present invention may be practiced without these specific details.

The present disclosure is to be considered as an exemplification of the invention and is not intended to limit the invention to the specific embodiments illustrated by the figures or description below.

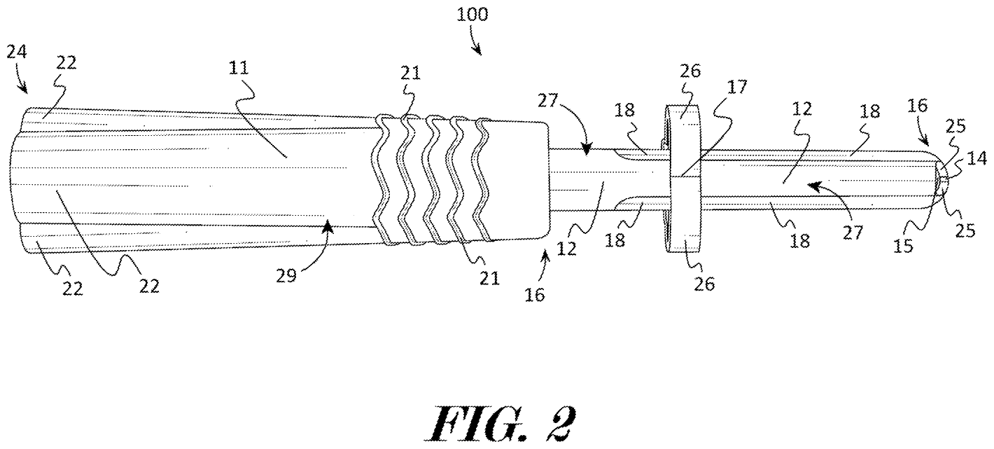

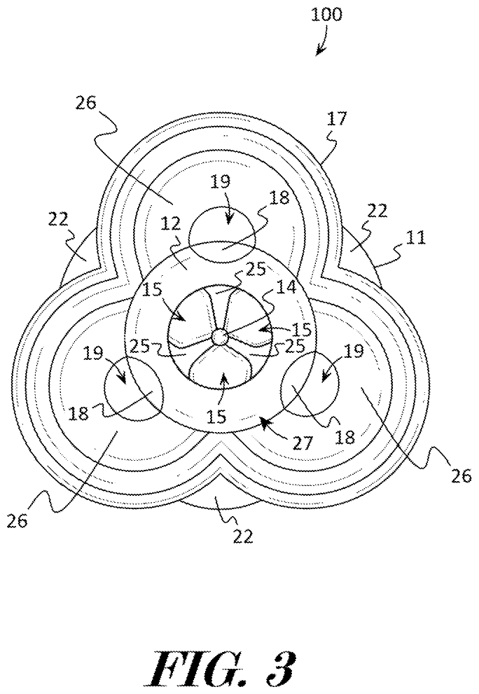

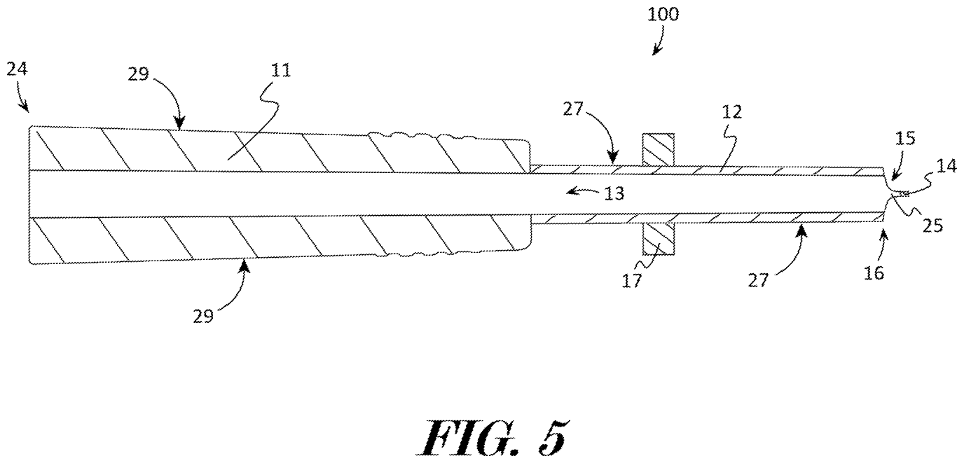

The present invention will now be described by example and through referencing the appended figures representing preferred and alternative embodiments. FIGS. 1-6 illustrate examples of a rectal insertion device (“the device”) 100 according to various embodiments. In some embodiments, the device 100 may comprise a body 11 that may be coupled to a shaft 12. The body 11 may include a distal end 24 and the shaft 12 may include a proximal end 16. A conduit 13 may extend through the body 11 and the shaft 12 between the distal end 24 and the proximal end 16. The shaft 12 may include a nose 14 which may be opposingly positioned to the body 11. One or more shaft apertures 15 may be disposed in the nose 14, and each shaft aperture 15 may be in communication with the conduit 13. A stopper 17 may be coupled to the shaft 12, and the stopper 17 may surround and extend above a portion of the shaft 12. The shaft 12 may include one or more grooves 18 which may be depressed into the shaft 12, and the stopper 17 may include one or more stopper apertures 19 which may extend through the stopper 17. Preferably, each groove 18 may be aligned with a stopper aperture 19 as perhaps best shown in FIG. 3. The device 100 may be used by inserting the shaft 12 into the anus of an individual with the insertion limited by the stopper 17. Gas may exit the individual by passing through the conduit 13 and/or by passing in a groove 18 and out through a stopper aperture 19.

In preferred embodiments, the device 100 or one or more elements of the device 100 may be made from or comprise a polymer or plastic material, such as polyethylene (PE), polypropylene (PP) and polyvinyl chloride (PVC), polycarbonate, nylon, Poly(methyl methacrylate) (PMMA) also known as acrylic, natural rubber, such as vulcanized rubber, synthetic rubber, such as styrene-butadiene rubber. In further preferred embodiments, the device 100 or one or more elements of the device 100 may be made from or comprise a flexible biocompatible material include man-made polymers, such as silicones, poly (ethylene), poly (vinyl chloride), polyurethanes, polypropylene, Polymethylmetacrylate, Polytetrafluoroethylene, Polyamide, Polyethylenterephthalate, Polyethersulfone, polylactides, Polyetherimide, and natural polymers, such as collagen, gelatin, elastin, silk, and polysaccharide. In other embodiments, the device 100 or one or more elements of the device 100 may be made from or comprise any material which may be suitable for contacting internal and/or external portions of the body including combinations of materials.

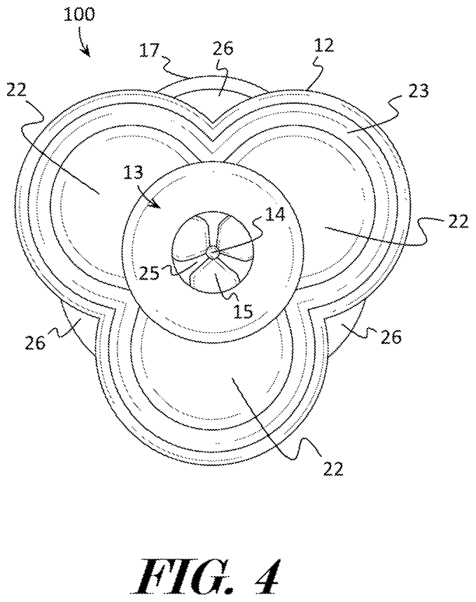

In some embodiments, the body 11 may be generally cylindrical in shape with portions of the conduit 13 extending centrally through the body 11. Likewise, in some embodiments, the shaft 12 may be generally cylindrical in shape with portions of the conduit 13 extending centrally through the shaft 12. The body 11 may have a distal end 24 and the shaft 12 may have a proximal end 16. The body 11 and the shaft 12 may be coupled together so that the distal end 24 and proximal end 16 are opposingly positioned to each other, and the conduit 13 may extend the length of the body 11 and the shaft 12 between the distal end 24 and proximal end 16 as perhaps best shown in FIG. 4. In preferred embodiments, the body 11 and the shaft 12 may both comprise a generally elongated shape. In further preferred embodiments, an elongated body 11 may be coupled to an elongated shaft 12 so that the body 11 and shaft 12 are in a generally linear orientation. In preferred embodiments, the shaft 12 may comprise a shaft surface 27 that may be generally smooth to ease insertion and removal from the anus.

In some embodiments, a conduit 13 may comprise a generally elongated cylindrical shape. In other embodiments, a conduit 13 may comprise a generally elongated triangular prism shape, rectangular prism shape, hexagonal prism shape, or any other shape including combinations of shapes.

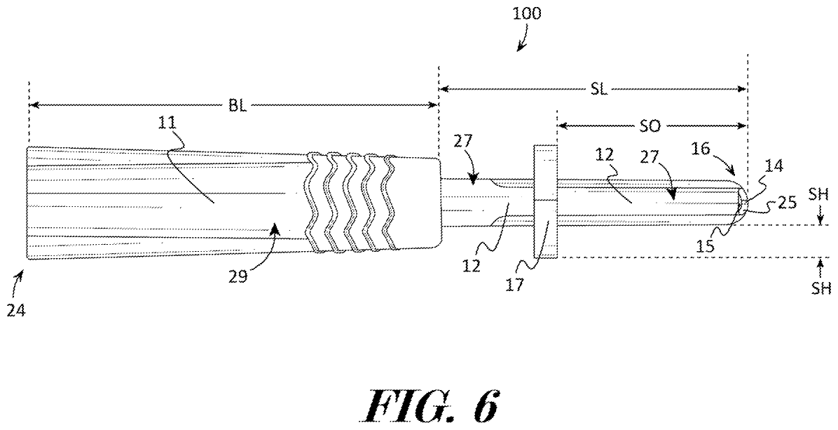

As shown in FIG. 6, the device 100 may comprise a body length dimension (BL) which may describe the length of the body 11. In some embodiments, a body length dimension (BL) may be approximately between 1.0 and 6.0 inches. In preferred embodiments, a body length dimension (BL) may be approximately between 2.0 and 4.0 inches. The device 100 may also comprise a shaft length dimension (SL) which may describe the length of the shaft 12, nose arms 25, and nose 14. In some embodiments, a shaft length dimension (SL) may be approximately between 0.75 and 6.0 inches. In preferred embodiments, a shaft length dimension (SL) may be approximately between 1.0 and 4.0 inches.

In some embodiments, the shaft 12 may include one or more grooves 18 which may be depressed into the shaft surface 27 of the shaft 12. The one or more grooves 18 may allow gas trapped by the anal sphincter to exit the individual optionally by entering a groove 18 and passing between the groove 18 and the anal wall to exit the anus (optionally by also passing through a stopper aperture 19). A groove 18 may be configured in any size and shape and may be positioned anywhere on the shaft 12. In preferred embodiments, one or more grooves 18 may extend from the nose 14 along all or portions of the shaft 12 towards the body 11 so that the groove(s) 18 may be parallel with the conduit 13. In further preferred embodiments, each groove 18 may be aligned with a stopper aperture 19. In still further preferred embodiments, a groove 18 may extend through the stopper aperture 19 of stopper 17.

In some embodiments, the body 11 may be configured as a handle which may be used to position the shaft 12 in and out of the anus of an individual. Optionally, the body 11 may comprise texturing 21 which may be suitable for increasing a user's ability to grip the body 11. Texturing 21 may be positioned anywhere on the body 11, such as between the portion of the body 11 that is coupled to the shaft 12 and the distal end 24 of the body 11 that is farthest from the shaft 12, and may be configured in any shape and size. Generally, texturing 21 may comprise a protrusion and/or depression that may extend above or below the surface 29 of the body 11. For example, the device 100 may comprise texturing 21 that may be configured as a rib-like protrusion that may extend above the body surface 29 and that extends around or encircles the body 11.

In some embodiments, portions of the body 11 distal to the shaft 12 may be configured with a lobed shape. For example, the body 11 may comprise three body lobes 22 which may be in a generally triangular orientation which the conduit 13 extending centrally through the body lobes 22. In further embodiments, the body 11 may comprise one or more body channels 23. Generally, a body channel 23 may form a depression in the distal end 24 of the body 11. Optionally, a body channel 23 may be in communication with the conduit 13. In further embodiments, the body 11 may comprise three triangularly oriented body lobes 22, with each body lobe 22 comprising a body channel 23 so that the body channels 23 may be generally triangularly oriented.

In some embodiments, the device 100 may include a nose 14 which may be coupled to the proximal end 16 of the shaft 12 so as to be opposingly positioned to the body 11. Generally, the nose 14 may form the portions of the device 100 that are farthest from the distal end 24 of the body 11. When the device 100 is in use, portions of the shaft 12 that are proximate to the nose 14 and the nose 14 may be configured to be inserted into the anus of an individual so that at least the nose 14 may be positioned past the anal sphincter. In this manner, gas trapped by the anal sphincter may be allowed to exit the individual optionally by entering the nose 14 and passing through the conduit 13 to exit the device 100.

The nose 14 may function as the initial element of the device 100 that contacts and enters a user's body, such as a blunted or rounded tip. In some embodiments, a nose 14 may be positioned away from the proximal end 16 and coupled to the proximal end 16 via one or more nose arms 25. In preferred embodiments, a nose 14 may be coupled to the proximal end 16 of the shaft 12 via three nose arms 25.

In some embodiments, the nose 14 may comprise one or more shaft apertures 15, and each shaft aperture 15 may be in communication with the conduit 13. In some embodiments, the device 100 may comprise one or more nose arms 25 which may bound or form all or portions of a shaft aperture 15. In further embodiments, all or portions of a shaft aperture 15 may be bounded or formed by portions of the proximal end 16, portions of one or more nose arms 25, and/or portions of the nose 14. For example, the device 100 may comprise three shaft apertures 15 and a nose 14 which may be coupled to the proximal end 16 of the shaft 12 via a first nose arm 25, a second nose arm 25, and a third nose arm 25 so that each shaft aperture 15 is formed and bounded by portions of two nose arms 25, portions of the nose 14, and portions of the proximal end 16.

The nose arms 25 may separate the rectal tissue as the nose 14 and shaft 12 are inserted into the anus to facilitate insertion. In some embodiments, the nose 14 may comprise three nose arms 25 which may bound three shaft apertures 15, and the shaft apertures 15 may be generally triangular in shape. In other embodiments, the nose 14 may comprise one, two, four, five, six, seven, or more nose arms 25 which may bound two, four, five, six, seven, eight, or more shaft apertures 15, and the shaft apertures 15 may be configured in any shape and size.

In some embodiments, the device 100 may comprise a stopper 17 which may be coupled to the shaft 12. A stopper 17 may extend away from or above the shaft 12 to contact portions of the individual that are external to the anus. In this manner, the stopper 17 may function as a barrier that limits how far the nose 14 and shaft 12 may be inserted into the rectal canal. A stopper 17 may be configured in any size and shape. In preferred embodiments, the stopper 17 may surround or encircle a portion of the shaft 12.

As shown in FIG. 6, the device 100 may comprise a stopper offset dimension (SO) which may describe the closest distance of a stopper lobe 22 (and therefore the stopper 17) is positioned away from the nose 14. In some embodiments, a stopper offset dimension (SO) may be approximately between 0.5 and 3.0 inches. In preferred embodiments, a stopper offset dimension (SO) may be approximately between 0.7 and 2.0 inches.

In preferred embodiments, the stopper 17 may comprise one or more stopper lobes 26, such as three stopper lobes 26, which may be arranged in a generally triangular orientation around portions of the shaft 12. In further preferred embodiments, the stopper lobes 26 may contact each other and may be positioned to encircle the shaft 12. In alternative embodiments, one or more stopper lobes 26 may not contact another stopper lobe 26.

As shown in FIG. 6, the device 100 may comprise a stopper height dimension (SH) which may describe the distance that the highest part of a stopper lobe 22 (and therefore the stopper 17) extends above the shaft surface 27. In some embodiments, a stopper height dimension (SH) may be approximately between 0.15 and 1.5 inches. In preferred embodiments, a stopper height dimension (SH) may be approximately between 0.15 and 0.75 inches.

In some embodiments, device 100 may comprise one or more stopper apertures 19 which may be positioned in the stopper 17 and which may extend through the stopper 17. Gas and other objects may exit the anus of an individual having the device 100 inserted into their anus via a stopper aperture 19 without requiring the gas and other objects from having to pass through the conduit 13. Preferably, all or portions of a stopper aperture 19 may be formed into or by a stopper lobe 26 and/or portions of the shaft 12, such as a groove 18.

In further embodiments, the stopper 17 may comprise one or more stopper lobes 26, and each stopper lobe 26 may comprise one or more stopper apertures 19. For example, a stopper 17 may comprise three stopper lobes 26 and each stopper lobe 26 may comprise a stopper aperture 19. A stopper aperture 19 may be configured in any size and shape, and a stopper aperture 19 may be positioned anywhere on a stopper 17. In preferred embodiments, a stopper aperture 19 may be generally positioned at a junction between the stopper 17 and the shaft 12.

In preferred embodiments, device 100 may comprise an equal number of grooves 18 and stopper apertures 19, and each groove 18 may be in communication with a respective stopper aperture 19. For example, the device 100 may comprise a first groove 18, a second groove 18, and a third groove 18, and a stopper 17 which includes a first stopper aperture 19, a second stopper aperture 19, and a third stopper aperture 19. The first groove 18 may be in communication with the first stopper aperture 19, the second groove 18 may be in communication with the second stopper aperture 19, and the third groove 18 may be in communication with the third stopper aperture 19.

Optionally, the stopper 17 may comprise one or more stopper channels 28 which may be positioned anywhere on the stopper 17. A stopper channel 28 may generally form a depression or recess into portions of the stopper 17, and a stopper channel 28 may be configured in any size and shape. In preferred embodiments, a first stopper channel 28 may be positioned on the side of the stopper 17 that is closer to the nose 14, and a second stopper channel 28 may be positioned on the side of the stopper 17 that is closer to the body 11. In further preferred embodiments, a stopper channel 28 may comprise a partially annular or ring shape so as to generally match the partially annular or ring shape of a body lobe 22 and more preferably each body lobe 22.

While some exemplary shapes and sizes have been provided for elements of the device 100, it should be understood to one of ordinary skill in the art that the body 11, shaft 12, conduit 13, and any other element described herein may be configured in a plurality of sizes and shapes including “T” shaped, “X” shaped, square shaped, rectangular shaped, cylinder shaped, cuboid shaped, hexagonal prism shaped, triangular prism shaped, or any other geometric or non-geometric shape, including combinations of shapes. It is not intended herein to mention all the possible alternatives, equivalent forms or ramifications of the invention. It is understood that the terms and proposed shapes used herein are merely descriptive, rather than limiting, and that various changes, such as to size and shape, may be made without departing from the spirit or scope of the invention.

Additionally, while some materials have been provided, in other embodiments, the elements that comprise the device 100 may be made from or may comprise durable materials such as aluminum, steel, other metals and metal alloys, wood, hard rubbers, hard plastics, fiber reinforced plastics, carbon fiber, fiber glass, resins, polymers or any other suitable materials including combinations of materials. Additionally, one or more elements may be made from or comprise durable and slightly flexible materials such as soft plastics, silicone, soft rubbers, or any other suitable materials including combinations of materials. In some embodiments, one or more of the elements that comprise the device 100 may be coupled or connected together with heat bonding, chemical bonding, adhesives, clasp type fasteners, clip type fasteners, rivet type fasteners, threaded type fasteners, other types of fasteners, or any other suitable joining method. In other embodiments, one or more of the elements that comprise the device 100 may be coupled or removably connected by being press fit or snap fit together, by one or more fasteners such as hook and loop type or Velcro® fasteners, magnetic type fasteners, threaded type fasteners, sealable tongue and groove fasteners, snap fasteners, clip type fasteners, clasp type fasteners, ratchet type fasteners, a push-to-lock type connection method, a turn-to-lock type connection method, a slide-to-lock type connection method or any other suitable temporary connection method as one reasonably skilled in the art could envision to serve the same function. In further embodiments, one or more of the elements that comprise the device 100 may be coupled by being one of connected to and integrally formed with another element of the device 100.

Although the present invention has been illustrated and described herein with reference to preferred embodiments and specific examples thereof, it will be readily apparent to those of ordinary skill in the art that other embodiments and examples may perform similar functions and/or achieve like results. All such equivalent embodiments and examples are within the spirit and scope of the present invention, are contemplated thereby, and are intended to be covered by the following claims.

What is claimed is:

1. A rectal insertion device for relieving gas in a human or animal, the device comprising:
a. a body coupled to a shaft, the body having a distal end and the shaft having a proximal end;
b. a conduit extending between the distal end of the body and the proximal end of the shaft;
c. a first shaft aperture formed into the proximal end of the shaft, the first shaft aperture in communication with the conduit; and
d. a stopper coupled to the shaft, the stopper extending above the shaft.

2. The device of claim 1, wherein the stopper encircles the shaft.

3. The device of claim 1, wherein the stopper includes a first stopper aperture.

4. The device of claim 3, wherein the stopper comprises a stopper lobe and wherein portions of the first stopper aperture is formed into the first stopper lobe.

5. The device of claim 1, further comprising a first groove depressed into the shaft.

6. The device of claim 5, wherein the stopper includes a first stopper aperture, and wherein the first groove is in communication with the first stopper aperture.

7. The device of claim 5, further comprising a second groove depressed into the shaft and a third groove depressed into the shaft, wherein the stopper includes a first stopper aperture, a second stopper aperture and a third stopper aperture, wherein the first groove is in communication with the first stopper aperture, wherein the second groove is in communication with the second stopper aperture, and wherein the third groove is in communication with the third stopper aperture.

8. The device of claim 1, wherein the body comprises texturing.

9. The device of claim 1, further comprising a nose coupled to the proximal end of the shaft via first nose arm.

10. The device of claim 9, wherein portions of the first shaft aperture are bounded by the first nose arm.

11. The device of claim 9, further comprising a second nose arm and a second shaft aperture.

12. A rectal insertion device for relieving gas in a human or animal, the device comprising:
a. a body coupled to a shaft, the body having a distal end and the shaft having a proximal end;
b. a conduit extending between the distal end of the body and the proximal end of the shaft;
c. a first shaft aperture formed into the proximal end of the shaft, the first shaft aperture in communication with the conduit;
d. a first groove depressed into the shaft; and
e. a stopper coupled to the shaft, the stopper extending above the shaft, wherein the stopper includes a first stopper aperture.

13. The device of claim 12, wherein the stopper encircles the shaft.

14. The device of claim 13, wherein the stopper comprises a stopper lobe and wherein portions of the first stopper aperture is formed into the first stopper lobe.

15. The device of claim 12, wherein the first groove is in communication with the first stopper aperture.

16. The device of claim 12, further comprising a second groove depressed into the shaft and a third groove depressed into the shaft, wherein the stopper includes a first stopper aperture, a second stopper aperture and a third stopper aperture, wherein the first groove is in communication with the first stopper aperture, wherein the second groove is in communication with the second stopper aperture, and wherein the third groove is in communication with the third stopper aperture.

17. The device of claim 12, wherein the body comprises texturing.

18. The device of claim 12, further comprising a nose coupled to the proximal end of the shaft via first nose arm.

19. The device of claim 18, wherein portions of the first shaft aperture are bounded by the first nose arm.

20. The device of claim 18, further comprising a second nose arm and a second shaft aperture.
